(19) United States
(12) Patent Application Publication
(10) Pub. No.: US 2012/0277803 A1
(43) Pub. Date: Nov. 1, 2012

(54) BONE PLATE & METHOD FOR MANUFACTURING

(75) Inventors: Rajesh Remesh, Memphis, TN (US); Mark Dace, Collierville, TN (US); Abhijit Raval, Memphis, TN (US); Jonathan Gant, Byhalia, MS (US); Clinton Jacob, Memphis, TN (US)

(73) Assignee: Warsaw Orthopedic, Inc., Warsaw, IN (US)

(21) Appl. No.: 13/093,946

(22) Filed: Apr. 26, 2011

(51) Int. Cl. A61B 17/80; B23P 11/00

(52) U.S. Cl. 606/289; 29/525.01; 606/286

(57) ABSTRACT

A method of manufacturing an orthopedic plate is disclosed. The method comprises the following steps: providing a stratum comprising a first surface for engaging bone and a second surface opposing said first surface, wherein the stratum comprises a fastener hole for receiving a fastener, wherein the fastener has a head and a shaft; providing a retaining element comprising a head and a shaft; creating a retaining hole for receiving the retaining element, wherein the retaining element extends through at least the second surface; placing the retaining element into the retaining hole to reach a fully-inserted position; cutting a portion of the head of the retaining element such that the head of the fastener can be inserted through the fastener hole and pass by the head of the retaining element to reach a fully-inserted position of the fastener. Also, an orthopedic plate made using the disclosed method is disclosed.

### FIELD OF INVENTION

The present invention is directed to systems for affixing a stratum to bone.

### BACKGROUND

The present disclosure relates to retaining mechanisms, and more particularly, systems for affixing a stratum to bone as well as methods of manufacturing the same.

### SUMMARY OF THE INVENTION

A method of manufacturing an orthopedic plate is disclosed. The method comprises the following steps: providing a stratum comprising a first surface for engaging bone and a second surface opposing said first surface, wherein the stratum comprises a fastener hole for receiving a fastener, wherein the fastener has a head and a shaft; providing a retaining element comprising a head and a shaft; creating a retaining hole for receiving the retaining element, wherein the retaining element extends through at least the second surface; placing the retaining element into the retaining hole to reach a fully-inserted position; cutting a portion of the head of the retaining element such that the head of the fastener can be inserted through the fastener hole and pass by the head of the retaining element to reach a fully-inserted position of the fastener. Also, an orthopedic plate made using the disclosed method is disclosed.

Further, an orthopedic plate that is not fully manufactured is disclosed. The orthopedic plate that is not fully manufactured comprises a stratum, a retaining element, and a retaining hole for receiving the retaining element. The stratum comprises a first surface for engaging bone and a second surface opposing said first surface, wherein the stratum comprises a fastener hole for receiving a fastener, wherein the fastener has a head and a shaft. The retaining element comprises a head and a shaft, wherein the retaining element extends through at least the second surface, and wherein the retaining element has been inserted into the retaining hole to reach a fully-inserted position.

Further, methods of implanting a spinal plate are disclosed.

### BRIEF DESCRIPTION OF THE DRAWINGS

FIG. 1 is a top isometric view of a stratum;

FIG. 2 is a top isometric view of a first retaining element;

FIG. 3 is a top isometric view of a stratum in cooperation with a first retaining element and a second retaining element;

FIG. 4 is another top isometric view of a stratum in cooperation with a first retaining element and a second retaining element;

FIG. 5 is another top isometric view of a stratum in cooperation with a first retaining element and a second retaining element;

FIG. 6 is another top isometric view of a stratum in cooperation with a first retaining element and a second retaining element;

FIG. 7 is a top isometric view of a spinal plate used to join three bone portions;

FIG. 8 is a top isometric view of a stratum;

FIG. 9 is a top isometric view of a retaining element that is not fully manufactured;

FIG. 10 is a top isometric view of an orthopedic plate that is not fully manufactured;

FIG. 11 is a bottom isometric view of the stratum of FIG. 10;

FIG. 12 is another top isometric view of the stratum of FIG. 10 after one of the retaining elements has been fully manufactured;

FIG. 13 is a top isometric view of the orthopedic plate of FIG. 10 after it has been fully manufactured; and

FIG. 14 is a top isometric view of an orthopedic plate that has been fully manufactured.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

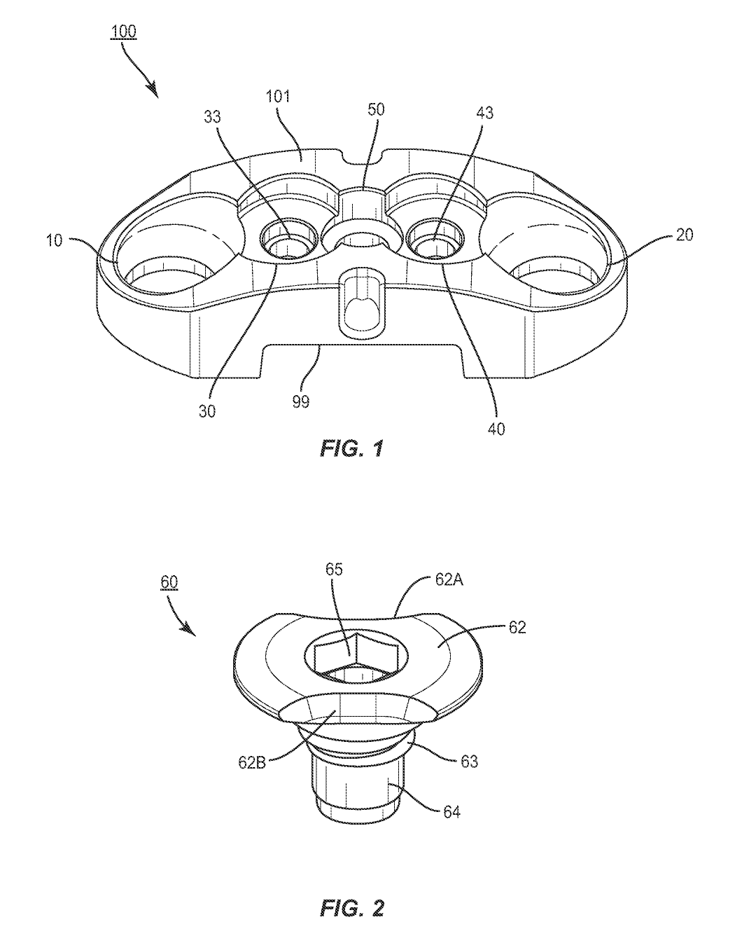

FIG. 1 shows a top isometric view of a stratum 100 comprising a first surface 99 for engaging bone and a second surface 101 opposing said first surface 99, wherein the stratum 100 comprises a first fastener hole 10 for receiving a first fastener and a second fastener hole 20 for receiving a second fastener. Each fastener (not shown) has a head and a shaft. In cases where the stratum 100 is a bone plate or a spinal plate, the fasteners (not shown) may be bone screws.

As shown in FIG. 1, the stratum 100 further comprises a first retaining hole 30 for receiving a first retaining element, wherein the first retaining hole 30 extends through at least the second surface 101. As shown in FIG. 1, the first retaining hole 30 extends through the second surface 101 and the first surface 99. Similarly, as shown in FIG. 1, the stratum 100 further comprises a second retaining hole 40 for receiving a second retaining element, wherein the second retaining hole 40 extends through at least the second surface 101. As shown in FIG. 1, the second retaining hole 40 extends through the second surface 101 and the first surface 99.

As shown in FIG. 1, the stratum 100 further comprises an interbody hole 50. Interbody hole 50 may be provided on a stratum 100 for alignment with an interbody device such as a spacer. For example, in addition to affixing the stratum 100 to bone by using fasteners, one may affix the stratum to an interbody device, for example, by inserting a fastener through interbody hole 50 and into the interbody device.

FIG. 2 shows a top isometric view of a first retaining element 60. As shown in FIG. 2, the first retaining element 60 comprising a head 62 and a shaft 64 and threads 63 on the shaft 64. As shown in FIG. 1, the first retaining hole 30 comprises threads 33 for receiving the first retaining element 60. Specifically, the threads 63 on the shaft 64 of the first retaining element 60 engage threads 33 of the first retaining hole 30. As shown in FIG. 2, the head 62 of the first retaining element 60 has two cut-outs 62A and 62B. Further, as shown in FIG. 2, the head 62 has an opening 65 for manipulation of the first retaining element 60 by a user with a tool such as a screw driver.

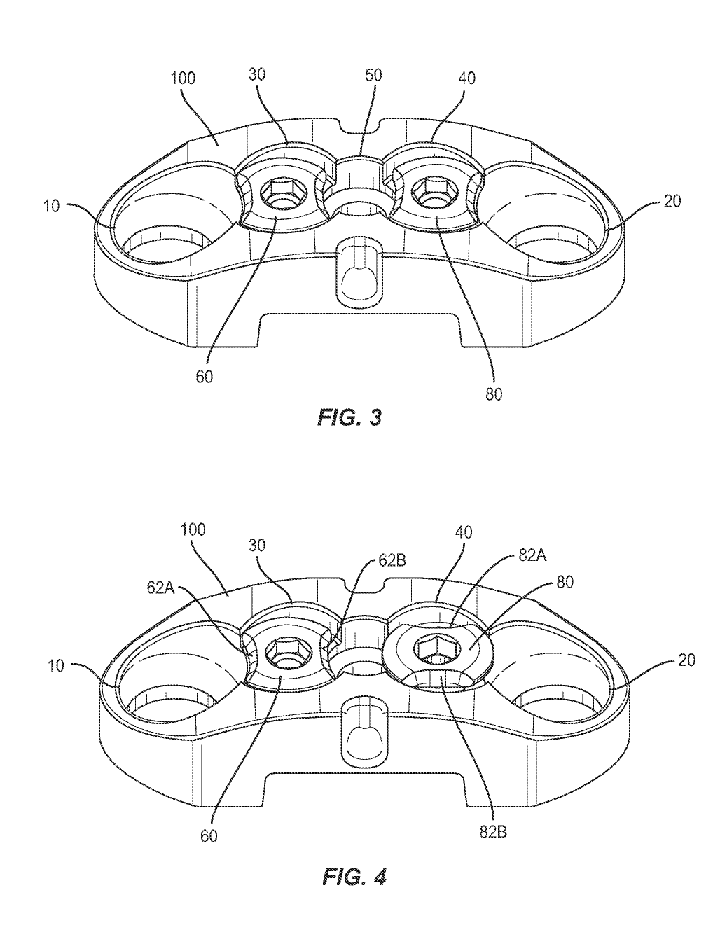

FIG. 3 shows a top isometric view of stratum 100 in cooperation with the first retaining element 60 and a second retaining element 80. Specifically, as shown in FIG. 3, the first retaining element 60 and the second retaining element 80 are situated in the first retaining hole 30 and the second retaining hole 40, respectively. Note that, as shown in FIG. 3, the second retaining element 80 has features similar to that of the first retaining element 60. As shown in FIG. 3, the first retaining element 60 is in a first position for allowing the first fastener to pass through the first fastener hole 10 and into bone adjacent the first surface 99 of the stratum 100. Specifically, the first retaining element 60 does not overlap or block the first fastener hole 10, thereby allowing the first fastener to pass through the first fastener hole 10. As shown in FIG. 3, the second retaining element 80 is in a first position for allowing the second fastener to pass through the second fastener hole 20 and into bone adjacent the first surface 99 of the stratum 100. Specifically, the second retaining element 80 does not overlap or block the second fastener hole 20, thereby allowing the second fastener to pass through the second fastener hole 20.

FIG. 4 shows another top isometric view of stratum 100 in cooperation with the first retaining element 60 and the second retaining element 80. Specifically, as shown in FIG. 4, however, the second retaining element 80 is in a second position for not allowing the second fastener to pass back out of the second fastener hole 20 and out of the bone adjacent the first surface 99 of the stratum 100. Specifically, if the second fastener (not shown) already has been inserted into the second fastener hole 20 to reach its fully-inserted position, the second retaining element 860 overlaps the second fastener hole 20 so as to prevent the second fastener from backing out of the second fastener hole 20 and bone through which it was inserted.

As in FIG. 3, FIG. 4 shows the first retaining element 60 in its first position such that cut-out 62A is adjacent the first fastener hole 10 and the first retaining element 60 does not overlap or block the first fastener hole 10, thereby allowing the second fastener to pass through the second fastener hole 20. That is, as shown in FIGS. 3 and 4, a first fastener may pass through the first fastener hole 10 and into bone to its fully-inserted position.

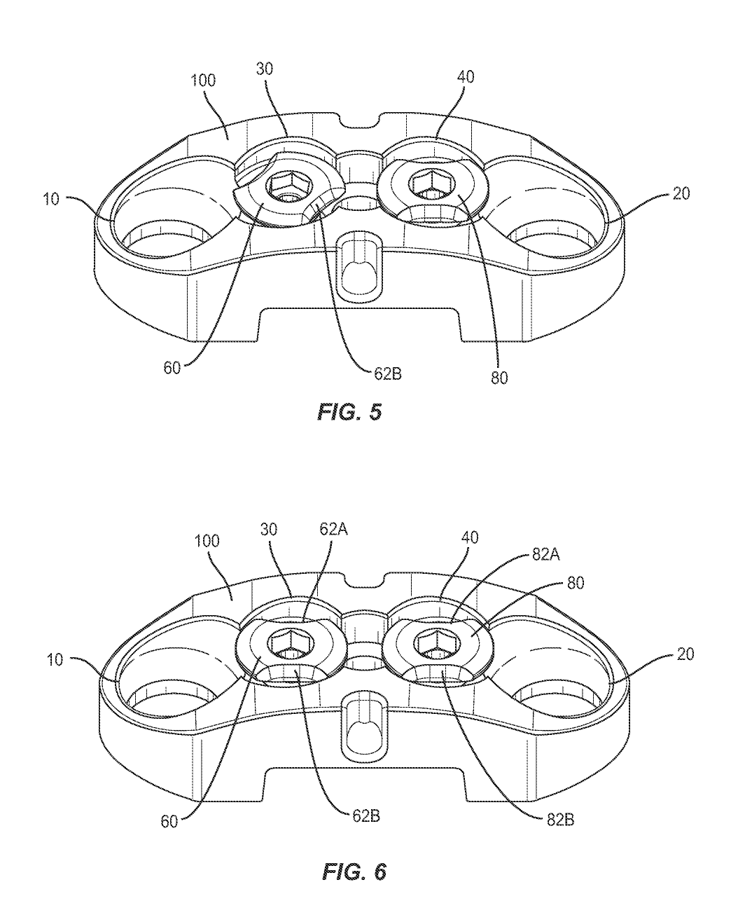

FIG. 5 shows another top isometric view of stratum 100 in cooperation with the first retaining element 60 and the second retaining element 80. Specifically, as shown in FIG. 5, however, the second retaining element 80 is still in its second position, whereas the first retaining element 60 is in between its first and second positions. As shown in FIG. 5, while the first retaining element 60 may prevent a first fastener from backing out of the first fastener hole 10, the first retaining element 60 is not in its second position, a position specifically designed to prevent a first fastener from backing out of the first fastener hole 10.

FIG. 6 shows another top isometric view of stratum 100 in cooperation with the first retaining element 60 and the second retaining element 80. Specifically, as shown in FIG. 6, the first retaining element 60 and the second retaining element 80 are each in their second positions to prevent the first and second fasteners from backing out of the first and second fastener holes 10 and 20, respectively.

In operation, one may use stratum 100 to join two portions of bone. One may place the stratum so that the first fastener hole 10 overlaps a first bone portion, and so that the second fastener hole 20 overlaps a second bone portion. Second, while the first and second retaining elements 60 and 80 are in their respective first positions, one may insert bone fasteners into each of the first and second fastener holes 10 and 20 until they reach their fully-inserted positions in the bone. Third, one may rotate the first and second retaining elements 60 and 80 from their respective first positions to their respective second positions, which may be done, for example, by turning the first and second retaining elements 60 and 80 in a clockwise direction until they reach their respective fully-inserted positions. With the first and second retaining elements 60 and 80 in their respective second positions, the fasteners may not back out of fastener holes 10 and 20 and the bone.

A stratum for accomplishing the aforementioned steps is provided to a user, for example, as it is shown in FIG. 3. The stratum would have been manufactured so that the threads 63 of the retaining element 60 are “timed” with the threads 33 of the first retaining hole 30 so that after the first fastener is inserted through the stratum 100, the first retaining element 60 may be rotated clockwise 90 degrees and it would reach its second position. This precise timing or spacing of the threads, however, is challenging, time consuming and often requires scrapping of some material or product. In addition, manufacturing in this way requires additional time to ensure the quality of such timing. Further, manufacturing in this way may result in a variation of the first and a second positions that while allowable and within tolerances, are less than ideal. That is, the first position of the first retaining element 60 may not be exactly at 0 degrees—or vertical—as shown in FIG. 3, but may vary by a few degrees. Similarly, the second first position of the first retaining element 60 may not be exactly at 90 degrees—or horizontal—as shown in FIG. 6, but may vary by a few degrees.

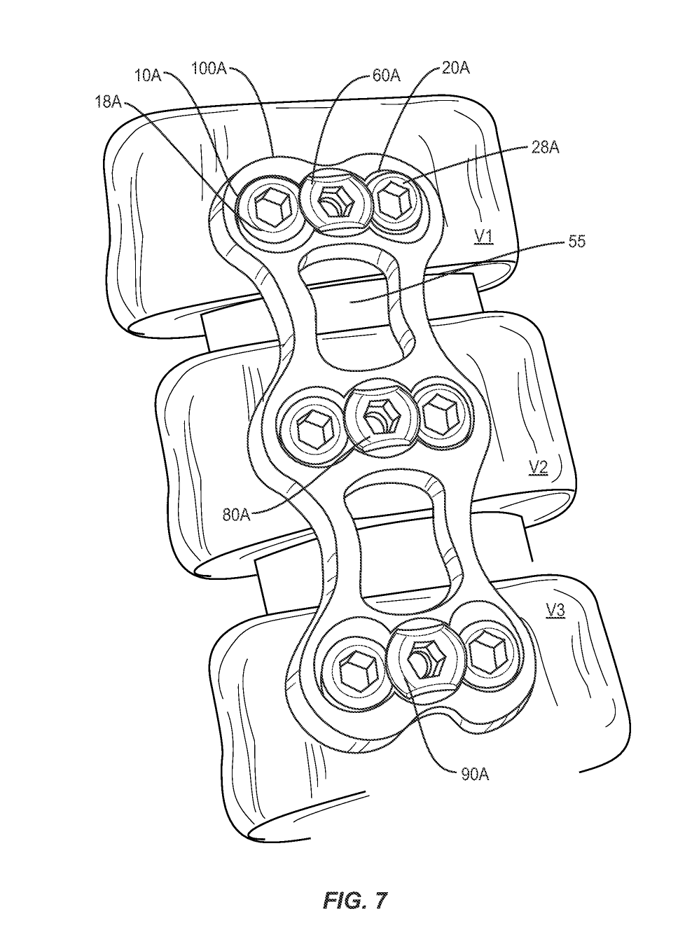

FIG. 7 shows a top isometric view of another stratum 100A, which is a spinal plate 100A used to join three bone portions or vertebral bodies V1, V2 and V3. Stratum 100A comprises a first fastener hole 10A for receiving a first fastener 18A and a second fastener hole 20A for receiving a second fastener 28A. As shown in FIG. 7, the first retaining element 60A is in its second position to prevent the first and second fasteners 18A and 28A from backing out of the first and second fastener holes 10A and 20A, respectively. Similarly, a second retaining element 80A and a third retaining element 90A are in their second positions to prevent their respective fasteners from backing out of their respective fastener holes. Further, as shown in FIG. 7, note that there is an intervertebral disc space shown between vertebral bodies V1 and V2 and an interbody device 55 or spacer is situated in this disc space.

A new method of manufacturing an orthopedic plate is disclosed. FIG. 8 shows a top isometric view of a stratum 200 comprising a first surface 199 for engaging bone and a second surface 201 opposing said first surface 199, wherein the stratum 200 comprises a first fastener hole 110 for receiving a first fastener and a second fastener hole 120 for receiving a second fastener. Each fastener (not shown in FIG. 8) has a head and a shaft. In cases where the stratum 200 is a bone plate or a spinal plate, the fasteners (for example, such as shown in FIG. 7) may be bone screws.

As shown in FIG. 8, the stratum 200 further comprises a first retaining hole 130 for receiving a first retaining element, wherein the first retaining hole 130 extends through at least the second surface 201. As shown in FIG. 8, the first retaining hole 130 extends through the second surface 201 and the first surface 199. Similarly, as shown in FIG. 8, the stratum 200 further comprises a second retaining hole 140 for receiving a second retaining element, wherein the second retaining hole 140 extends through at least the second surface 201. As shown in FIG. 8, the second retaining hole 140 extends through the second surface 201 and the first surface 199.

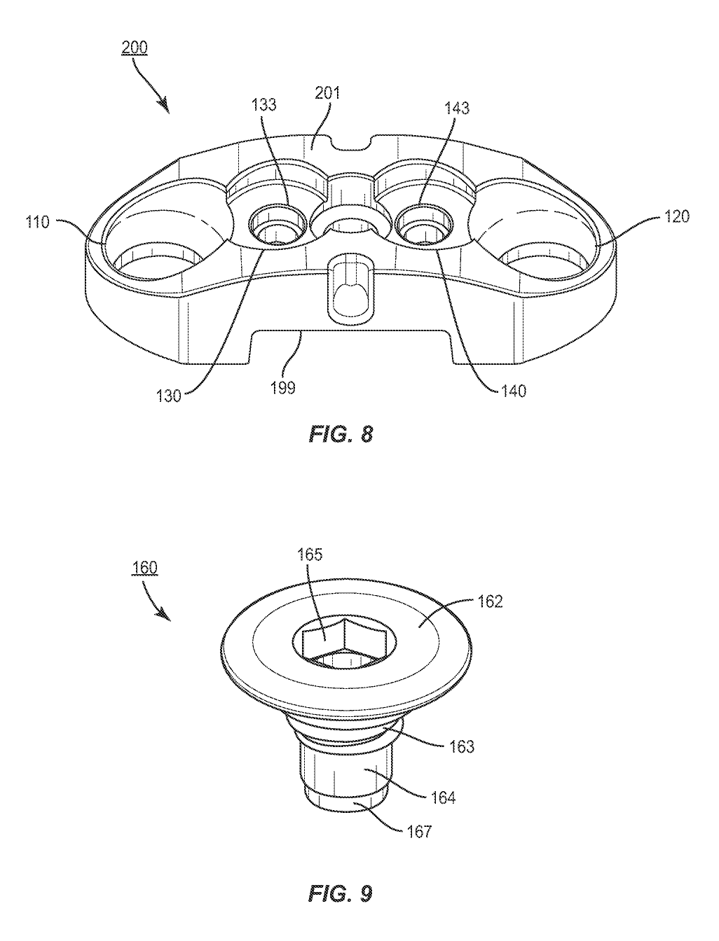

FIG. 9 shows a top isometric view of a first retaining element 160 that is not fully manufactured. As shown in FIG. 9, the first retaining element 160 comprising a head 162 and a shaft 164 and threads 163 on the shaft 164. As shown in FIG. 9, the first retaining hole 130 comprises threads 133 for receiving the first retaining element 160. Specifically, the threads 163 on the shaft 164 of the first retaining element 160 engage the threads 133 of the first retaining hole 130. As shown in FIG. 9, as opposed to that shown in FIG. 2, the head 162 of the first retaining element 160 does not have any cut-outs. Further, as shown in FIG. 9, the head 162 has an opening 165 for manipulation of the first retaining element 160 by a user with a tool such as a screw driver. In addition, as shown in FIG. 9, the shaft 164 has a proximal end adjacent the head 162 and a distal end 167 on the opposite end of the shaft 164.

At this stage of manufacturing, the stratum 200 and the retaining element 160 is provided. The stratum comprises a first surface 199 for engaging bone and a second surface 201 opposing said first surface 199. The stratum 200 further comprises a fastener hole 110 for receiving a fastener, wherein the fastener has a head and a shaft. The retaining element 160 comprises a head 162 and a shaft 164. The next step in the method of manufacturing is that of placing the retaining element 160 into the retaining hole 130 to reach a fully-inserted position, for example. For example, turning the retaining element 160 in a clockwise direction until it cannot be turned any farther may place the retaining element 160 in its fully-inserted position.

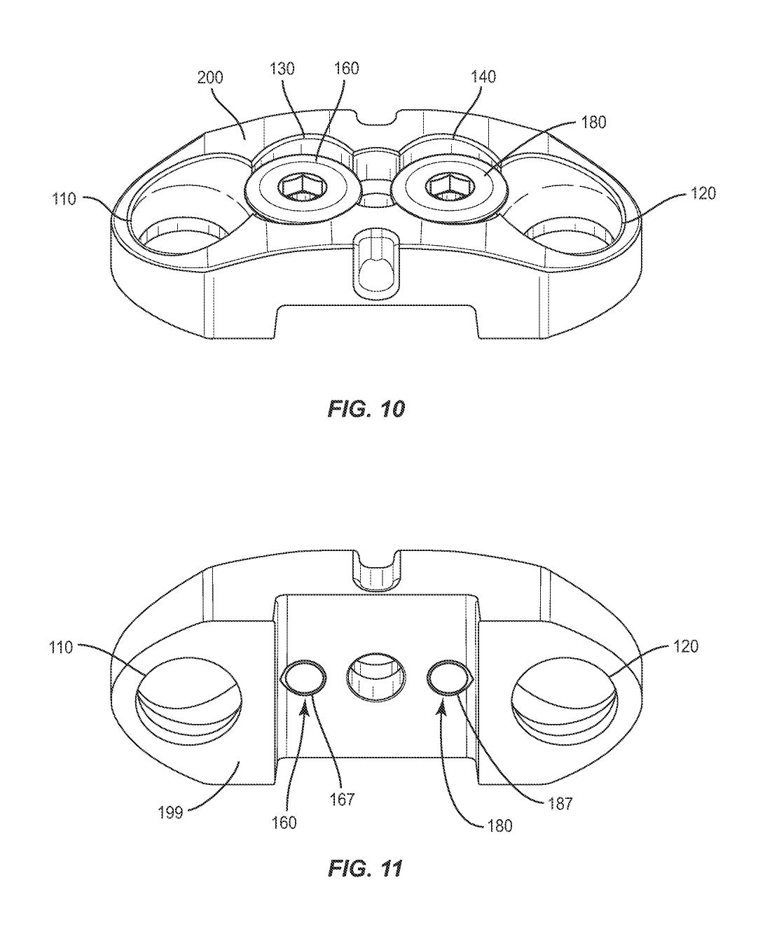

FIG. 10 shows a top isometric view of an orthopedic or spinal plate that has not been fully manufactured, or more specifically, of stratum 200 after retaining element 160 has been fully inserted into retaining hole 130 and after a second retaining element 180 that has not been fully manufactured has been inserted into a second retaining hole 140. As shown in FIG. 10, note that first and second fastener holes 110 and 120 are partially covered by the heads of the first and second retaining elements 160 and 180, respectively. In the embodiment disclosed herein, after the retaining elements 160 and 180 have been inserted into the retaining holes 130 and 140, respectively, to their fully-inserted positions, distal ends 167 and 187 of the respective shafts 164 and 184 of the respective retaining elements 160 and 180 extend past the first surface 199 of the stratum 200.

Further, note that the stratum shown in FIG. 10 may be considered an orthopedic plate that is not fully manufactured. As shown in FIG. 10, the orthopedic plate comprises a stratum 200, a retaining element 160 and a retaining hole 130 for receiving the retaining element 160. As shown in FIG. 10, the stratum 200 comprises a first surface 199 for engaging bone and a second surface 201 opposing said first surface 199, wherein the stratum 200 comprises a fastener hole 110 for receiving a fastener, wherein the fastener has a head and a shaft. As shown in FIG. 10, the retaining element 160 comprises a head 162 and a shaft 164. Further, as shown in FIG. 10, the retaining element 130 extends through at least the second surface 201, and the retaining element 160 has been inserted into the retaining hole 130 to reach a fully-inserted position. Also, the retaining element 160 may pass through both the second surface 201 and the first surface 199. In addition, as shown in FIG. 10, the head 162 of the retaining element 160 has a perimeter having a circular shape. When stratum 200 is an orthopedic plate, for example, the fastener may be a bone screw.

The next step in the method of manufacturing is that of staking the retaining element 160 to the stratum by securing the distal end 167 of the shaft 164 of the retaining element 160 to the first surface 199 of the stratum 200. FIG. 11 shows a bottom isometric view of the stratum 200 after the step of staking has taken place. Specifically, FIG. 11 shows the distal end 167 of the shaft 164 of the first retaining element 160 staked to the first surface 199 of the stratum 200. Similarly, FIG. 11 shows the distal end 187 of the shaft of the second retaining element 180 staked to the first surface 199 of the stratum 200. Staking may be accomplished in a variety of ways. With respect to the retaining element 160, for example, staking may be achieved when the distal end 167 of the shaft 164 of the retaining element 160 is fitted or crimped to the first surface 199 such that the retaining element 160 cannot be unscrewed or pulled back out of the retaining hole 130.

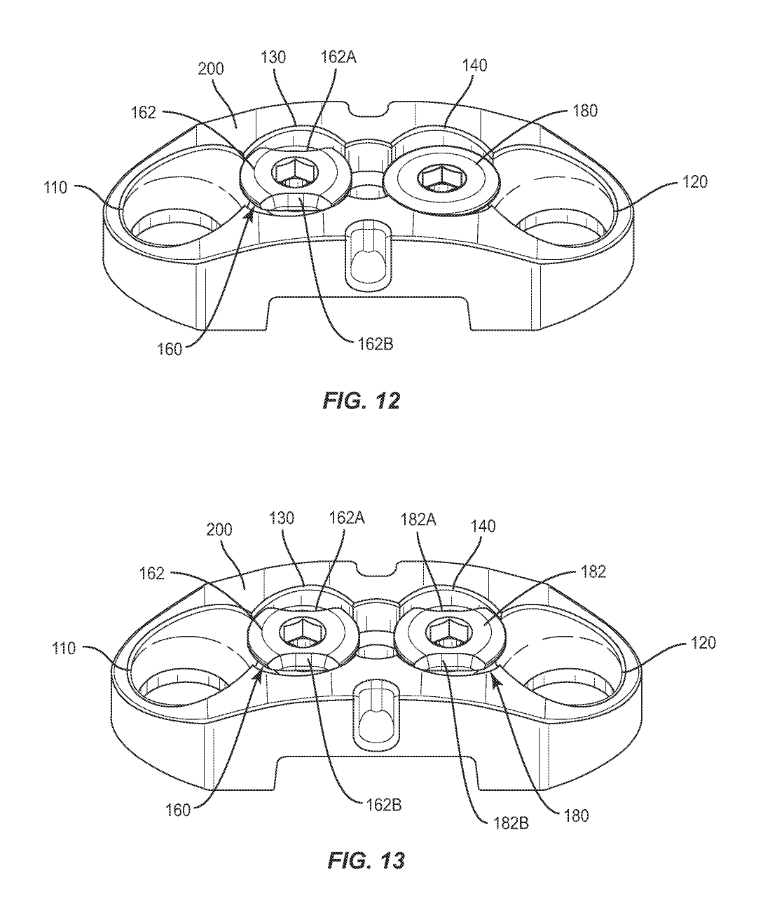

The next step in the method of manufacturing is that of cutting a portion of the head 162 of the retaining element 160 such that the head of the fastener can be inserted through the fastener hole 110 and pass by the head 162 of the retaining element 160 to reach a fully-inserted position of the fastener. FIG. 12 shows a top isometric view of the stratum 200 after the step of cutting the head 162 of the first retaining element 160 has taken place. Specifically, FIG. 12 shows the cut-outs 162A and 162B of head 162, which remain after cutting has taken place. When provided, the head 162 of the retaining element 160 had a perimeter having a circular shape (as shown in FIG. 10), and after the step of cutting, sections of the head 162 of the retaining element 160 has been removed so that the perimeter of the head 162 of the retaining element 160 no longer has a circular shape. As shown in FIG. 12, cutting the head of the second retaining element 180 has not yet occurred.

FIG. 13 shows a top isometric view of the stratum 200 after the step of cutting the head 182 of the second retaining element 180 has taken place. Specifically, FIG. 13 shows the cut-outs 182A and 182B of head 182, which remain after cutting has taken place. Thus, FIG. 13 shows a top isometric view of the stratum 200 after manufacturing is complete. As shown in FIG. 13, the retaining elements 160 and 180 are each in their second positions and the respective threads of the retaining elements 160 and 180 and their corresponding retaining holes 130 and 140 already have been timed to match. Thus, as shown in FIG. 13, the retaining elements 160 and 180 are situated substantially horizontal. To allow for fasteners to be placed through the stratum 200, the retaining elements 160 and 180 are rotated 90 degrees in a counterclockwise direction so that a top isometric view of the stratum 200 would look like that shown in FIG. 3.

The term “substantially” as used herein may be applied to modify any quantitative representation which could permissibly vary without resulting in a change in the basic function to which it is related. For example, a retaining element may be considered substantially horizontal in the second position even if it is not aligned at exactly 90 degrees, i.e., even though a retaining element is not aligned at exact exactly 90 degrees, it still may prevent a fastener from backing out of a stratum.

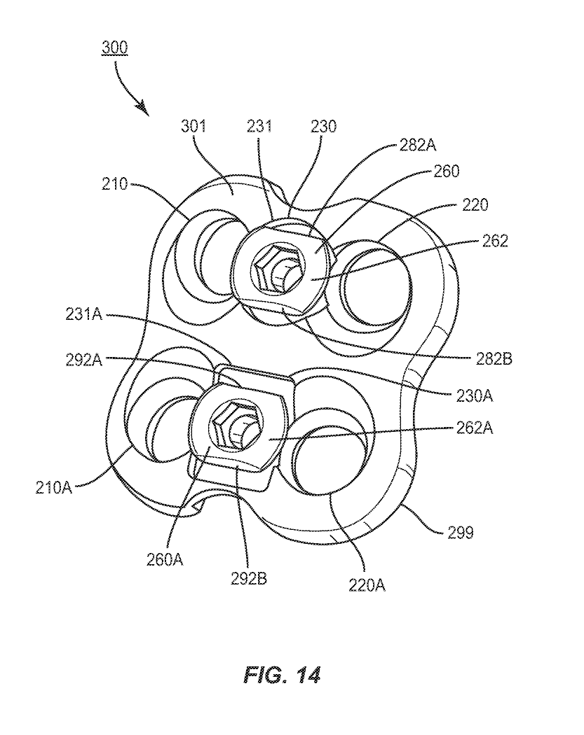

The step of cutting may be accomplished by a variety of methods. One such method, for example, is machining, and more specifically, milling. FIG. 14 shows a top isometric view of a stratum 300 after the step of cutting the heads of retaining elements 260 and 260A has taken place. Specifically, FIG. 14 shows a different stratum or spinal plate that has different fastener and retaining element arrangements than those previously described. Also, as shown in FIG. 14, stratum 300 comprises first and second fastener holes 210 and 220, with a retaining element 260 between these two fastener holes. Stratum 300 further comprises third and fourth fastener holes 210A and 220A, with a retaining element 260A between these two fastener holes. Note that retaining element 260 resides in retaining hole 230, whereas retaining element 260A resides in retaining hole 230A.

Further, as shown in FIG. 14, stratum 300 comprises a first surface 299 and a second surface 301. As shown in FIG. 14, retaining hole 230 has an outer perimeter 231, which is a recess in the second surface 301 and retaining hole 230A has an outer perimeter 231A or recess in the second surface 301. As shown in FIG. 14, outer perimeter 231 of retaining hole 230 has arcuate shapes that can accommodate the pre-cut arcuate shape of the retaining elements 260 and 260A before they are cut. For example, as shown in FIG. 9, the head 162 of retaining element 160 has a circular-shaped perimeter. Before they are cut, the heads of retaining elements 260 and 260A of FIG. 14 may have a shape similar to retaining element 160 of FIG. 9.

The outer perimeter of a retaining hole, however, may have a shape other than that shown for outer perimeter 231. For example, as shown in FIG. 14, outer perimeter 231A of retaining hole 230A has a shape larger than that of outer perimeter 231. Outer perimeter 231A may make it easier for one to cut the respective heads of retaining elements 260 and 260A.

In addition, FIG. 14 shows that cut-outs in retaining elements may have a variety of shapes as long as they accomplish their function. As FIG. 14 shows stratum 300 after the cutting step has taken place, retaining element 260 has cut-outs 282A and 282B, and retaining element 260A has cut-outs 292A and 292B. Note that cut-outs 282A, 282B, 292A and 292B have a linear shape, whereas previously-described cut-outs 62A, 62B, 82A and 82B have an arcuate shape. These shapes for the cut-outs described herein are not exhaustive as other shapes are possible that will still accomplish the required function.

In the embodiments described here, the various stratum or spinal plates may be made of a variety of biocompatible materials (metal or non-metal), including but not limited to, Titanium Alloys, commercially available Titanium, stainless steel, polyetheretherketone (“PEEK”), cobalt chrome (“CoCr”), polyetherketoneketone (“PEKK”), ultra high molecular weight polyethylene (“UHMWPE”), polyethylene, shape memory metals, other polymers or any combination of such materials. Similarly, the retaining elements and/or the fasteners may be made of the same materials. Also, any suitable materials know in the art may work for each of these elements.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. Furthermore, as used herein, the terms components and modules may be interchanged. It is understood that all spatial references, such as “superior,” “inferior,” “anterior,” “posterior,” “outer,” “inner,” and “perimeter” are for illustrative purposes only and can be varied within the scope of the disclosure.

What is claimed:

1. A method of manufacturing an orthopedic plate, the method comprising the steps of:
providing a stratum comprising a first surface for engaging bone and a second surface opposing said first surface, wherein the stratum comprises a fastener hole for receiving a fastener, wherein the fastener has a head and a shaft;
providing a retaining element comprising a head and a shaft;
creating a retaining hole for receiving the retaining element, wherein the retaining element extends through at least the second surface;
placing the retaining element into the retaining hole to reach a fully-inserted position;
cutting a portion of the head of the retaining element such that the head of the fastener can be inserted through the fastener hole and pass by the head of the retaining element to reach a fully-inserted position of the fastener.

2. The method of claim 1, wherein the fastener hole is a first fastener hole and the stratum further comprises a second fastener hole.

3. The method of claim 1, wherein the fastener is a bone screw.

4. The method of claim 1, wherein when provided, the head of the retaining element has a perimeter having a circular shape, and after the step of cutting, a section of the head of the retaining element has been removed so that the perimeter of the head of the retaining element no longer has a circular shape.

5. The method of claim 1, wherein retaining element extends through both the second surface and the first surface.

6. The method of claim 5, wherein after the retaining element has been inserted into the retaining hole to its fully-inserted position, a distal end of the shaft of the retaining element extends past the first surface.

7. The method of claim 6, further comprising the step of staking the retaining element to the stratum by securing the distal end of the shaft of the retaining element to the first surface of the stratum.

8. A orthopedic plate that is not fully manufactured, the plate comprising:
a stratum comprising a first surface for engaging bone and a second surface opposing said first surface, wherein the stratum comprises a fastener hole for receiving a fastener, wherein the fastener has a head and a shaft;
a retaining element comprising a head and a shaft;
a retaining hole for receiving the retaining element, wherein the retaining element extends through at least the second surface, and wherein the retaining element has been inserted into the retaining hole to reach a fully-inserted position.

9. The plate of claim 8, wherein the head of the retaining element has a perimeter having a circular shape.

10. The plate of claim 8, wherein the fastener is a bone screw.

11. The plate of claim 8, wherein the retaining element extends through both the second surface and the first surface.

12. The plate of claim 11, wherein a distal end of the shaft of the retaining element extends past the first surface.

13. The plate of claim 11, wherein a distal end of the shaft of the retaining element is secured to the first surface of the stratum.

14. An orthopedic plate manufactured by a method comprising the steps of:
providing a stratum comprising a first surface for engaging bone and a second surface opposing said first surface, wherein the stratum comprises a fastener hole for receiving a fastener, wherein the fastener has a head and a shaft;
providing a retaining element comprising a head and a shaft;
creating a retaining hole for receiving the retaining element, wherein the retaining element extends through at least the second surface;
placing the retaining element into the retaining hole to reach a fully-inserted position;
cutting a portion of the head of the retaining element such that the head of the fastener can be inserted through the fastener hole and pass by the head of the retaining element to reach a fully-inserted position of the fastener.

15. The plate of claim 14, wherein the fastener hole is a first fastener hole and the stratum further comprises a second fastener hole.

16. The plate of claim 14, wherein the fastener is a bone screw.

17. The plate of claim 14, wherein when provided during manufacturing, the head of the retaining element has a perimeter having a circular shape, and after the step of cutting, a section of the head of the retaining element has been removed so that the perimeter of the head of the retaining element no longer has a circular shape.

18. The plate of claim 14, wherein the retaining element extends through both the second surface and the first surface.

19. The plate of claim 14, wherein after the retaining element has been inserted into the retaining hole to its fully-inserted position, a distal end of the shaft of the retaining element extends past the first surface.

20. The plate of claim 19, further comprising the step of staking the retaining element to the stratum by securing the distal end of the shaft of the retaining element to the first surface of the stratum.
